(19) United States
(12) Patent Application Publication
(10) Pub. No.: US 2020/0330265 A1
(43) Pub. Date: Oct. 22, 2020

(54) Cold Block for Decreasing Neuropathy During Chemotherapy

(71) Applicants: Mindy Cody, West Coldwell, NJ (US); Margaret Femandez, West Coldwell, NJ (US)

(72) Inventors: Mindy Cody, West Coldwell, NJ (US); Margaret Femandez, West Coldwell, NJ (US)

(21) Appl. No.: 16/873,254

(22) Filed: Mar. 5, 2020

Related U.S. Application Data
(60) Provisional application No. 62/813,821, filed on Mar. 5, 2019.
(60) Provisional application No. 62/847,374, filed on May 14, 2019.

(51) Int. Cl. A61F 7/10

(52) U.S. Cl. CPC A61F 7/103; A61F 2007/0037

(57) ABSTRACT

A cold block for cooling an extremity comprises a container defining an internal chamber. A recess is defined in the container. The recess is surrounded, in part, by the internal chamber. The recess is configured to receive an extremity of a person. In use, in one example, the internal chamber of the cold block is filled with fluid, such as water, and frozen. In another example the internal chamber contains a gel that may be cooled or frozen. A patient undergoing chemotherapy may place a respective extremity in one or more cold blocks to decrease neuropathy.

### RELATED APPLICATIONS

The present application claims the benefit of U.S. Provisional Patent Application No. 62/813,821, which was filed on Mar. 5, 2019, and the present application claims the benefit of U.S. Provisional Patent Application No. 62/847,374, which was filed on May 14, 2019, both of which are incorporated by reference herein.

### FIELD OF THE INVENTION

Cold blocks for cold treatment of extremities, such as hands and feet, and, more particularly, a plastic container filled with ice for receiving an extremity during chemotherapy to decrease neuropathy.

### BACKGROUND OF THE INVENTION

Cancer patients being treated with chemotherapy often suffer from chemotherapy-induced peripheral neuropathy, which is caused by chemotherapy medications travelling through the body during and after treatment. Chemotherapy-induced peripheral neuropathy causes pain, numbness, tingling, and other symptoms in the hands and feet of patients. From about 30% to about 40% of chemotherapy patients suffer from chemotherapy induced peripheral neuropathy. In some cases, symptoms may end soon after chemotherapy ends. In other cases it can take 3 to 6 months or longer to dissipate after chemotherapy ends. Symptoms may never end for some patients. People being treated for breast cancer with platinum and taxane chemotherapy treatments are particularly vulnerable.

It has been found that cryotherapy may help control the symptoms of chemotherapy induced peripheral neuropathy. Patients apply cold packs filled with cold water or ice or gel during their chemotherapy treatments, but cold packs often reach room temperature before their chemotherapy treatment ends. To overcome this problem, patients often bring multiple cold packs in coolers. Coolers filled with ice and cold packs may be very heavy and difficult for cancer patients to bring to treatment. In addition, because of the shape of typical ice packs and gel packs, they are difficult to maintain in a position covering the tips of the fingers and toes, which are particularly vulnerable to chemotherapy induced peripheral neuropathy.

Cold gloves and socks are also used by chemotherapy patients during treatment. These products do not stay cold long enough for a patient to ice their fingers and toes during the entire length of many chemotherapy sessions. Patients are therefore required to buy multiple pairs of gloves and socks and bring them to their sessions in order to continually ice their hands and feet during infusion of chemotherapy medications.

### SUMMARY OF THE INVENTION

An improved cryotherapy system that is more convenient for patients is needed during chemotherapy.

In accordance with an embodiment of the invention, a cold block for cooling an extremity comprises a container defining an internal chamber configured to contain a cold material and a recess defined in the container. The recess is defined by an exterior surface of the container. The recess is surrounded, in part, by the internal chamber, and is configured to receive an extremity of a person to cool the extremity when a cold material is within the internal chamber.

In accordance with another embodiment, a cold block for cooling an extremity is disclosed comprising a bottom wall, a top wall, and at least one side wall between the bottom wall and the top wall. The bottom wall, the top wall and the at least one side wall are connected to form a container having an internal chamber configured to receive a coolable material. One of the at least one side wall defines a recess extending into the internal chamber. The recess is configured to receive an extremity of a person. The recess has a bottom wall separated from the bottom wall of the container, such that a portion of the internal chamber is adjacent the bottom wall. A top wall is separated from the top wall of the container such that a portion of the internal chamber is adjacent the top wall. A first side wall is separated from the at least one side wall of the container, such that a portion of the internal chamber is adjacent the first side wall. A second side wall is separated from the at least one side wall of the container, such that a portion of the internal chamber is adjacent the second side wall. The second side wall faces the first side wall. A backwall is separated from the at least one side wall, such that a portion of the internal chamber is adjacent to the backwall. When cold or frozen material is within the internal chamber, the material is adjacent to the bottom wall, the top wall, and the first side wall, the second side wall and the backwall of the recess.

In accordance with another embodiment of the invention, a method of using a container defining an internal chamber and a recess defined in an exterior wall of the container is disclosed. The recess is surrounded, in part, by the internal chamber. The method comprises freezing a fluid and/or gel in the container, placing a portion of an extremity in the recess in the container, and cooling the portion of the extremity placed in the recess. The method further comprises undergoing chemotherapy for a period of time, while the extremity is in the recess for at least a portion of the period of time. The method may further comprise filling the internal chamber with water prior to freezing.

### BRIEF DESCRIPTION OF THE FIGURES

FIG. 1 is a front perspective view of a cold block, in accordance with an embodiment of the invention;

FIG. 2 is a left cross-sectional view of the cold block of FIG. 1;

FIG. 3 is a top view of the cold block of FIG. 1;

FIG. 4 is a left cross-sectional view of the cold block of FIG. 1, with a foot inserted into a recess in the cold block;

FIG. 5 is a left cross-sectional view of the cold block of FIG. 1, with a hand inserted into a recess in the cold block;

FIG. 6 is a vertical cross-sectional view of a hand in a recess of another embodiment of the invention;

FIG. 7 is a vertical cross-sectional view of a hand in a recess of another embodiment of the invention; and

FIG. 8 is a top view of another embodiment of the invention, for use with a foot.

### DESCRIPTION OF PREFERRED EMBODIMENTS

A cold block 10 for use by patients undergoing chemotherapy is disclosed. In one embodiment, a recess is provided in a container for containing a fluid that can be frozen or cooled, such as water or a gel, to receive at least a portion of an extremity of a patient so that the portion of the extremity is at least partially surrounded by cold or frozen fluid. Cooling one or more extremities is believed to decrease neuropathy in the extremity.

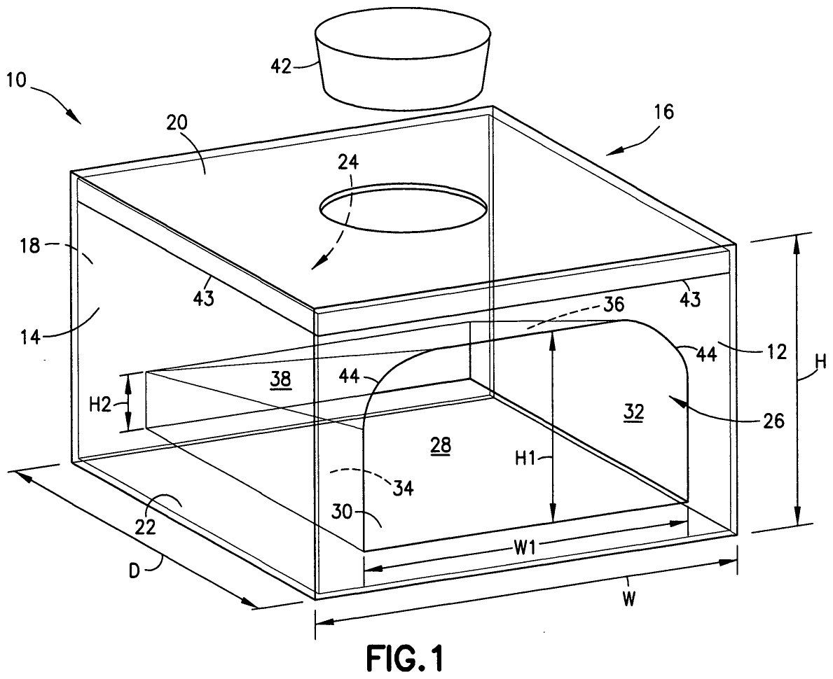

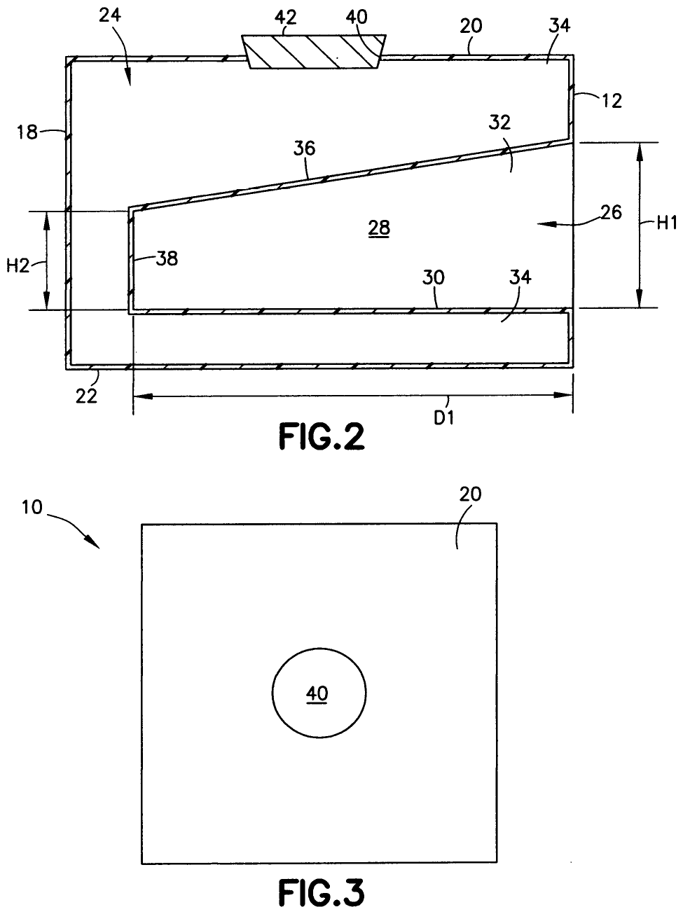

FIG. 1 is a front perspective view of a cold block 10, in accordance with an embodiment of the invention. FIG. 2 is a left cross-sectional view of FIG. 1. The cold block 10 includes a front wall 12, a left side wall 14, a right side wall 16, a rear wall 18, a top wall 20, and a bottom wall 22. The corners of the cold block 10 may be rounded.

The cold block 10 may be made of silicone, plastic, or other water-tight material, for example. The cold block 10 may also comprise multi-layers, such as aluminum and polyethylene layers reinforced with nylon, for example. While the cold block 10 of FIG. 1 is rectangular, the cold block 10 may have other shapes, such as cubic or cylindrical, for example. A plastic cold block may be made by molding, such as injection molding or blow molding, for example. The internal surfaces of the front wall 12, the left side wall 14, the right side wall 16, the rear wall 18, the top wall 20, and the bottom wall 22 are connected along their edges to define a container having an internal chamber 24, which is also shown in the left cross-sectional view of FIG. 2. The internal chamber 24 is water-tight in order to contain water or other fluid, or gel, that is to be frozen or cooled. A plastic cold block may be made by molding, such as injection molding or blow molding, for example.

The front wall 12 defines an entry 26 to a recess 28 of the cold block 10. The recess 28 has a bottom wall 30, a right side wall 32, a left side wall 34, a top wall 36, and a rear wall 38. The corners of the recess 28 may be curved and the open internal chamber may taper toward the rear wall 38. The recess 28 is configured to receive a hand or a foot of a patient, which rests on the bottom wall 30 of the recess. The recess 28 is shown in phantom in FIG. 1 and is shown in cross-section in FIG. 2. Tapering the top wall 36 downwardly toward the rear wall 38 brings the top wall, and the ice or other cold or frozen material in the internal chamber 24, behind the top wall, closer to the hand and finger tips, or foot and toes, of the user.

The top wall 20 of the cold block 10 includes an entry 40 into the internal chamber 24. FIG. 3 is a top view of the cold block 10, showing the entry 40. The entry 40 enables the internal chamber 24 to be filled with water. The entry 40 is closed with a plug 42, such as a rubber plug, for example, with a shape matching the shape of the entry 40. The entry 40 may be round or oval, for example. The entry 40 is shown closed by the plug 42 in FIG. 2. The plug 42 may be separate from the cold block 10 or may be connected to the cold block 10 by a chain or rope (not shown), for example. The plug may also be of other materials. Other types of closure mechanisms may be used instead of the plug 42.

In another example, if water or other fluid is not to be removed from the cold block 10 between sessions, or if the cold block 10 is filled with fluid during manufacture, a permanent closure may be used.

A fill line 43 is shown on the exterior surface of the left side wall 14 and the front wall 12. The fill line 43 may also be provided on the left side wall 14, the right side wall 15, and the rear wall 18 of the cold block 10. Alternatively, the fill line 43 may be provided on the exterior surface of only one of the walls. Filling the cold block above the fill line 43 could push out the plug 42 and/or expand the walls of the cold block 10 due to the expansion of water on freezing. The fill line is positioned about one-quarter of the height H of the cold block below the top wall 20, in this example.

The cold block 10 may have different dimensions for use with hands or feet, of adults of different sizes and children. A person may identify the proper size based on a template on a package or on a website, for example. In one example, cold blocks 10 may be provided having different dimensions for a large adult, a medium size adult, and a youth, for example. Cold blocks of more different sizes, or fewer sizes may be available. A cold block 10 for use with a hand of a large adult may have a height H of 4¾ inches, a depth D of 6¾ inches, and a width W of 6 inches, for example. A cold block 10 for use with a hand of a medium size adult may have a height H of 3¾ inches, a depth D of 5¾ inches, and a width W of 6 inches, for example. A cold block 10 for use with a hand of a youth may have a height H of 2¾ inches, a depth of 4¾ inches, and a width of 5 inches, for example.

A cold block 10 for use with a foot of a large adult may have a height H of 5¾ inches, a depth D of 7¾ inches, and a width W of 6 inches, for example. A cold block 10 for use with a foot of a medium size adult may have a height H of 4¾ inches, a depth D of 7 inches, and a width W of 6 inches, for example. A cold block 10 for use with a foot of a youth may have a height H of 2¾ inches, a depth D of 4¾ inches, and a width W of 5 inches, for example. These dimensions are exemplary and cold blocks 10 in accordance with embodiments of the invention may be of different sizes.

In all examples, the height H1 of the entry 26 to the recess 28 may be 2 inches and the height of the rear wall 38 of the recess may be 1 inch, as shown in FIG. 1 and FIG. 2. In all examples, the width W1 of the entry 26 may be 4½ inches. The depth D1 of the recess 28 may be 5 inches for adult large and adult medium sizes, and 4 inches for a youth, for example, as is best shown in FIG. 2. The upper wall 36 of the recess 28 tapers downwardly from the entry 26 to the rear wall 38 of the recess. The corners 44 of the entry 26 may be curved. As above, the dimensions of the recess 28 are exemplary and the recess of cold blocks 10 in accordance with embodiments of the invention may be of different sizes.

The thickness of all walls in this example is one-eight (⅛) inch. The walls may be of other sizes, as well. In the embodiment shown in FIG. 1 and FIG. 2, spaces are provided between the top wall 20 of the cold block 10 and the upper wall 36 of the recess, the rear wall 18 of the cold block and the rear wall 38 of the recess, the left side wall 14 cold block and the left wall of the recess, the right side wall of the cold block and the right wall 32 of the recess, and the bottom wall 22 of the cold block and the bottom wall 30 of the recess. This enables ice to be formed on all sides of the recess 28, so that the hand or foot (including the fingers and toes), inserted into the recess is surrounded on its top, sides, and bottom by ice when the cold block is frozen and an extremity is inserted into the recess. This is not required, however.

In another example, a single cold block 10 could be made wide enough to accommodate two hands and another single cold block could be made wide enough to accommodate two feet, by making the cold block and the respective recess wider. It may be desirable to also make the cold block taller when two extremities are to be accommodated.

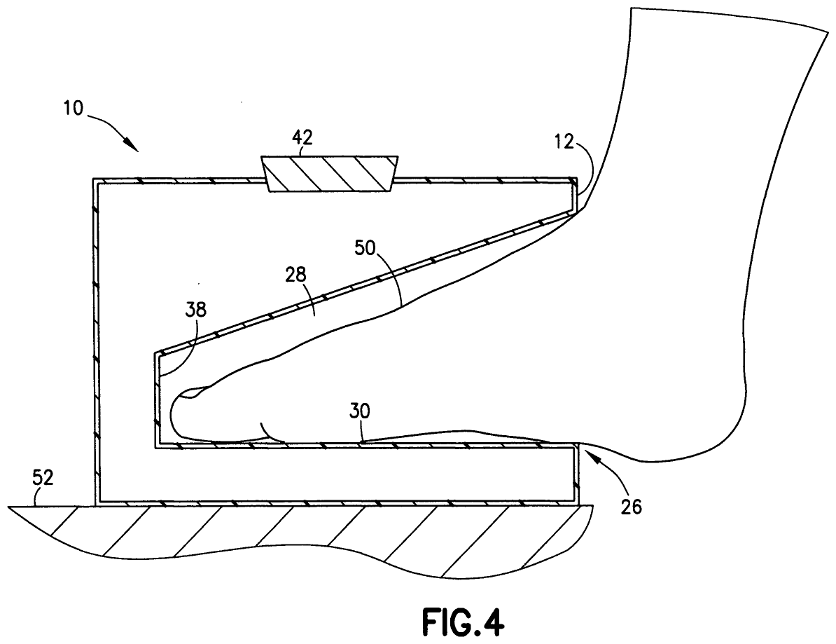

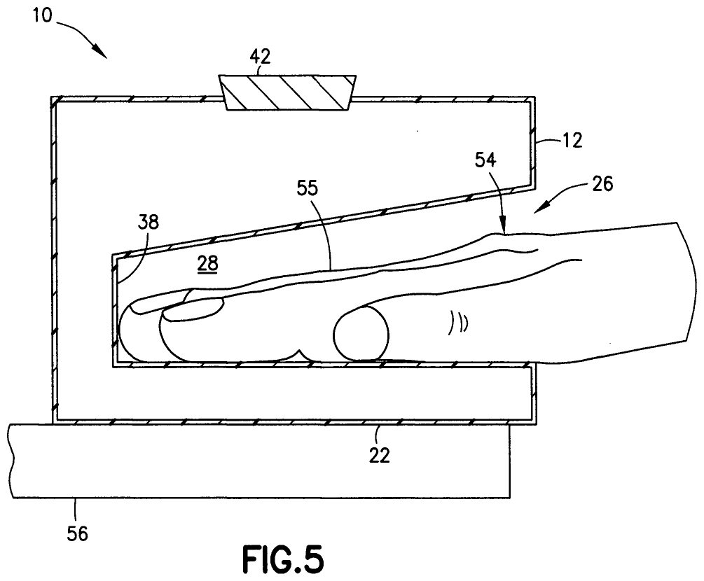

FIG. 4 is a cross-sectional view of a cold block 10 with a patient's foot 50 placed through the entry 26 of cold block 10 and into the recess 28 of the cold block 10. The cold block in FIG. 4 is placed on a floor 52. The patient's foot 50 rests against the bottom wall 30 of the recess 28. FIG. 5 is a cross-sectional view of a cold block 10 with a patient's hand 54 placed through the entry 26 of cold block 10 and into the recess 28. The cold block 10 in FIG. 5 is placed on a table 56. The patient's hand 54 rests against the bottom wall 30 of the recess 28. The middle finger 55 is shown directly adjacent to the rear wall 38 of the recess 28.

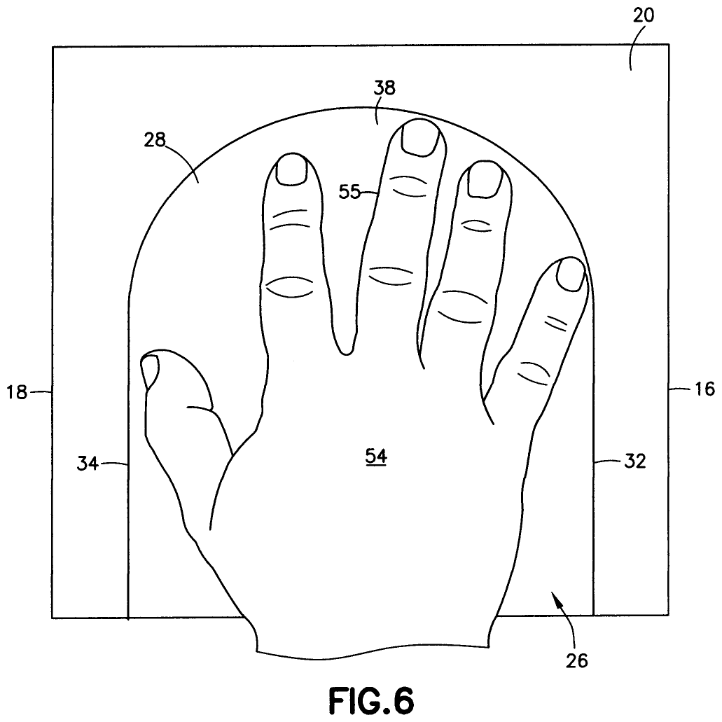

While the middle finger 55 in FIG. 5 is close to the rear wall 38 of the recess 28, the other fingers may not be as close. FIG. 6 is a vertical cross-sectional view through the recess 28 of another example of a cold block 10, where the back wall 38 is curved to follow the curvature of the all the fingers of a typical hand 54, in accordance with an embodiment of the invention. The fingertips of each of the fingers can therefore be closer to the back wall 38 and the ice behind the back wall. The remainder of the cold block 10 in FIG. 6 is the same as the cold block of FIG. 1 and is commonly numbered.

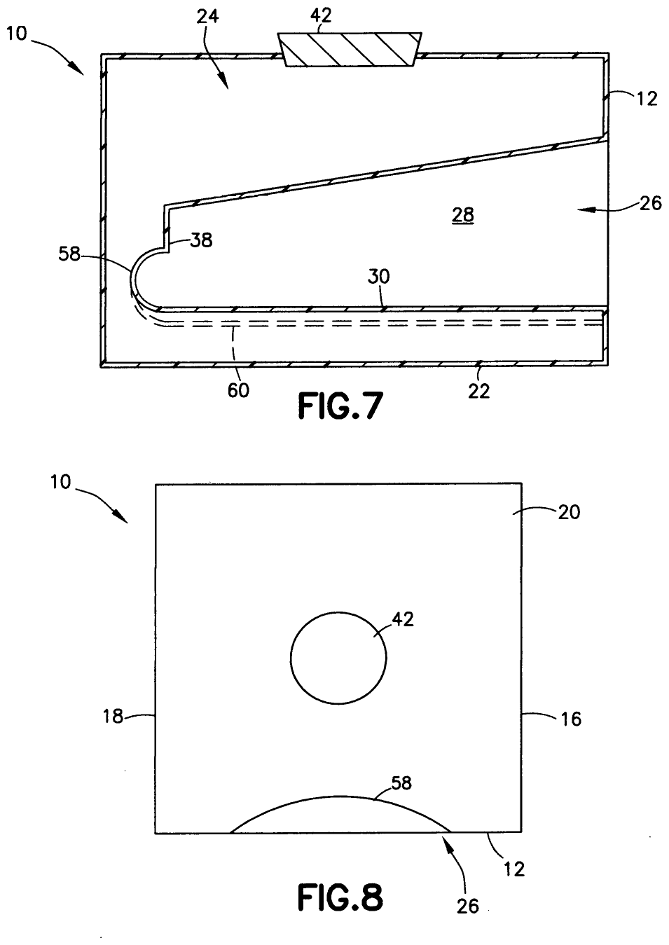

FIG. 7 is a vertical cross-sectional view through the recess 28 of another example of a cold block 10, where the back wall 38 includes an indent 58 to receive a fingertip or end of a toe, for better contact with the ice or other cold or frozen material in the internal chamber is curved to follow the curvature of the all the fingers of a typical hand 54, in accordance with another embodiment of the invention. Respective indents may be provided for some or all of the fingers or toes. Alternatively, a horizontal groove in the back wall 38 may be provided to receive several fingertips or toes. The indent 58 or groove may be deep enough for the finger or toe to be inserted up to the knuckle, for example. In addition, a groove 60 may be provided in the bottom wall 22 to receive a portion of the hand, fingers, and/or fingertips, or foot, toes or tips of toes, as shown in phantom in FIG. 7. The remainder of the cold block is the same as in FIG. 1 and is similarly numbered.

FIG. 8 is a top view of another example of a cold block 10 for a foot, where the front wall of the cold block above the entry 26 of the recess 28 has a cutaway 58 to enable a foot to be inserted deeper into the recess, in accordance with another embodiment of the invention. The remainder of the cold block is the same as in FIG. 1 and is similarly numbered.

As is known in the art, gels that may be used for cooling may be made from hydroxyethyl cellulose, sodium polyacrylate, and/or vinyl-coated silica gel, for example, as is known in the art. An example of a gel is blue gel, which is used in 3M Reusable Cold Packs and which contains from about 60% to about 70% water, from about 20% to about 35% propylene glycol, and less than 5% sodium carboxymethyl cellulose. The gel may be placed in the cold block 10 during manufacture, for example. In another example, the cold pack 10 may be filled with dry gel material(s) during manufacture and the user may fill the cold block with fluid, such as water, to convert the material(s) into a gel when they are ready to use it. The cold block may then be placed into a freezer before use to cool or freeze the gel. Other coolable or freezable materials may also be used.

In use, the internal chamber 24 of a cold block 10 is filled with water through the entry 40, to the fill line 43. The entry 40 is then closed with the plug 42 and placed in a freezer of a refrigerator, for example. In one example, four cold blocks 24 may be filled and placed in the freezer, one for each extremity. The cold blocks 10 will freeze in from about 12 hours to about 24 hours, for example, depending on the size of the cold block. The cold block 10 may therefore be placed in the freezer in the day or evening before a chemotherapy treatment. In another example, if the cold block 10 is filled with fluid, such as water or a gel, during manufacture, then the cold block 10 need only be placed in a freezer and cooled or frozen.

The frozen or cold blocks 10 may be removed from the freezer and placed into a cooler before a patient leaves for a chemotherapy treatment. Cold blocks 10 of the dimensions above weigh from about two (2) pounds to about three (3) pounds, for example. The cold blocks 10 are removed from the cooler at the location of chemotherapy. Two cold blocks 10 may be placed on the floor to accommodate the patient's feet and two cold blocks may be placed on a table or tray in front of the patient to accommodate the patient's hands.

A patient may start icing about 15 minutes before a treatment starts, for example. The patient may periodically remove their extremity from the cold block 10 during the treatment, such as every twenty minutes for from about 5 minutes to about 10 minutes, for example. The cold blocks 10 will stay cold long enough for a chemotherapy session of 90 minutes including the 15 minutes pre-cooling, for example.

After the chemotherapy session is completed, if the cold block 10 is filled with ice, the water resulting from melting of the ice may poured out of the entry 40 removing the plug 42 and turning the cold block upside down. Alternatively, the user can bring the cold block 10 home without pouring out the water. The cold block 10 may then be put in the freezer at home to freeze for the next chemotherapy session. Similarly, If the cold block 10 is filled without another material, such as a gel, then the user can bring the cold block home and then put the cold block in the freezer to freeze or cool for the next chemotherapy session

Exemplary embodiments of the invention are described above. It would be understood to one of ordinary skill in the art that modifications may be made to the embodiments described above without going beyond the spirit and scope of the invention, which is defined in the following claims.

1. A cold block for cooling an extremity, comprising:
a container defining an internal chamber configured to contain a cold material; and
a recess defined in the container, the recess being defined by an exterior surface of the container, surrounded, in part, by the internal chamber, the recess being configured to receive an extremity of a person and cool the extremity when a cold material is within the internal chamber.

2. The cold block of claim 1, wherein the exterior surface defines an entry for a fluid through the exterior surface to the internal chamber.

3. The cold block of claim 2, further comprising:
a plug to close the entry.

4. The cold block of claim 1, wherein the container comprises:
a side wall, wherein the recess is defined in the side wall.

5. The cold block of claim 1, wherein:
the recess has a bottom wall defined, in part, by a portion of an exterior surface of the side wall of the container, the bottom wall configured to support an extremity when the extremity is received in the recess; and
a portion of the internal chamber is below the portion of the exterior surface of the side wall.

6. The cold block of claim 5, wherein:
the recess has a first side wall defined, in part, by a second portion of the exterior surface of the side wall of the container, the first side wall being configured to be adjacent to a first side of the extremity when the extremity is received in the recess;
a second portion of the internal chamber is adjacent the second portion of the exterior surface of the side wall;
the recess has a second side wall defined, in part, by a third portion of the exterior surface of the side wall of the container, opposite the second side wall, the second side wall being configured to be adjacent to a second side of the extremity opposite the first side when the extremity is received in the recess; and
a third portion of the internal chamber is adjacent the second side wall of the exterior surface of the side wall.

7. The cold block of claim 6, wherein:
the recess has a top wall defined, in part, by a fourth portion of the exterior surface of the side wall of the container, the top wall being configured to be above the extremity when the extremity is received in the recess; and
a fourth portion of the internal chamber is above the top wall of the exterior surface of the side wall.

8. The cold block of claim 7, wherein:
the recess has a back wall defined by a fifth portion of the exterior surface of the side wall of the container, the back wall being configured to be behind extremity when the extremity is received in the recess; and
a fifth portion of the internal chamber is adjacent to a front of the extremity when the extremity is received in the recess.

9. A cold block for cooling an extremity, comprising:
a bottom wall, a top wall, and at least one side wall between the bottom wall and the top wall, the bottom wall, the top wall and the at least one side wall being connected to form a container having an internal chamber configured to receive a coolable material;
one of the at least one side wall defining a recess extending into the internal chamber, the recess configured to receive an extremity of a person;
the recess having:
a bottom wall separated from the bottom wall of the container, such that a portion of the internal chamber is adjacent the bottom wall;
a top wall separated from the top wall of the container such that a portion of the internal chamber is adjacent the bottom wall;
a first side wall separated from the at least one side wall of the container, such that a portion of the internal chamber is adjacent the first side wall;
a second side wall separated from the at least one side wall of the container, such that a portion of the internal chamber is adjacent the second side wall, wherein the second side wall faces the first side wall, and
a backwall separated from the at least one side wall, such that a portion of the internal chamber is adjacent to the backwall;
wherein, when cold or frozen material is within the internal chamber, the cold material is adjacent to the bottom wall, the top wall, and the first side wall, the second side wall and the backwall of the recess.

10. The cold block of claim 9, wherein the at least one side wall of the container comprises:
a first pair of opposing side walls; and
a second pair of opposing side walls.

11. The cold block of claim 9, wherein the top wall of the container defines a top wall, wherein the entry is defined through the top wall, into the internal chamber;
the cold block further comprising a plug to close the entry.

12. The cold block of claim 9, wherein the top wall of the recess is downwardly tapered toward the backwall.

13. The cold block of claim 9, wherein the back wall includes at least one indent toward the internal chamber to receive a fingertip or a toe.

14. The cold block of claim 9, wherein the bottom wall includes a groove to receive at least a portion of an extremity.

15. The cold block of claim 9, wherein the at least one side wall of the container defining the recess includes a recessed region above the recess, configured to accommodate a portion of a leg proximate the foot.

16. A method of using a container defining an internal chamber that is fluid-tight and a recess defined an exterior wall of the container, the recess being surrounded, in part, by the internal chamber, the method comprising:
cooling a fluid and/or gel in the container;
placing a portion of an extremity in the recess in the container;
cooling the portion of the extremity placed in the recess; and
undergoing chemotherapy for a period of time, wherein the extremity is in the recess for at least a portion of the period of time.

17. The method of claim 16, further comprising:
filling the internal chamber with water;
wherein cooling comprises freezing the water.

18. The method of claim 16, comprising placing the extremity in the recess before undergoing chemotherapy.

19. The method of claim 16, comprising cooling the extremity in the recess by frozen fluid surrounding all sides of the portion of the extremity in the recess.

20. The method of claim 16, further comprising:
providing at least two containers; and
placing at least two extremities in a respective one of the at least two containers.
